(19) United States
(12) Patent Application Publication
(10) Pub. No.: US 2014/0163122 A1
(43) Pub. Date: Jun. 12, 2014

(54) PRODUCTION OF SATURATED HYDROCARBONS FROM SYNTHESIS GAS

(71) Applicants: Qingjie Ge, Dalian (CN); Xiangang Ma, Dalian (CN); Junguo Ma, Dalian (CN); Hengyong Xu, Dalian (CN)

(72) Inventors: Qingjie Ge, Dalian (CN); Xiangang Ma, Dalian (CN); Junguo Ma, Dalian (CN); Hengyong Xu, Dalian (CN)

(73) Assignees: BP P.L.C., London (GB); DALIAN INSTITUTE OF CHEMICAL PHYSICS, CHINESE ACADEMY OF SCIENCES, Dalian, Liaoning (CN)

(21) Appl. No.: 14/113,071

(22) PCT Filed: Apr. 19, 2012
(86) PCT No.: PCT/CN12/74330

§ 371 Date: Feb. 20, 2014

(30) Foreign Application Priority Data
Apr. 21, 2011 (CN) PCT/CN2011/000695

(51) Int. Cl. C07C 1/04; C07C 1/24

(52) U.S. Cl. CPC C07C 1/0485; C07C 1/24
USPC 518/726; 422/630

(57) ABSTRACT

An integrated process for the generation of saturated C₃ and higher hydrocarbons from carbon oxide(s) and hydrogen, includes the steps of: (a) feeding a gas feed stream including carbon oxide(s) and hydrogen to a two-stage reaction system comprising a first stage including a carbon oxide(s) conversion catalyst, where the feed stream is converted in the first stage to form an intermediate product stream, (b) feeding the intermediate product stream to a second stage including a dehydration/hydrogenation catalyst and (c) removing a product stream from the second stage, the product stream including saturated C₃ and higher hydrocarbons. The two-stage reaction system could exhibit a high activity and selectivity to C₃ and higher hydrocarbons, and the two stage reactions may be operated in different reaction conditions.

This invention relates to the production of saturated hydrocarbons from synthesis gas. Some examples of the invention relate to the production of liquefied petroleum gas from synthesis gas. Some aspects of the invention may also find application in relation to the production of liquid fuels for example gasoline. Some aspects of the invention may find application in relation to an integrated system for the production of saturated hydrocarbons.

In recent years, the dominance of natural gas and petroleum as feedstocks has diminished. New feedstocks such as tar sands, coal, biomass and municipal waste have been increasing in importance. The diversity of feedstocks has driven the development of synthesis gas (syngas) routes to replace conventional routes to hydrocarbons from natural gas and petroleum.

Liquefied petroleum gas (LPG), a general description of propane and butane, has environmentally relatively benign characteristics and widely been used as a so-called clean fuel. Conventionally, LPG has been produced as a byproduct of liquefaction of natural gas, or as a byproduct of refinery operations. LPG obtained by such methods generally consists of mainly propane and n-butane mixtures. Alternative sources for LPG would be desirable. Synthesis of LPG from syngas is potentially a useful route as it would allow for the conversion of diverse feedstocks, for example natural gas, biomass, coal, tar sands and refinery residues.

One synthesis route to hydrocarbons uses the Fischer-Tropsch synthesis reaction. However, this can be disadvantageous in that the product hydrocarbons will follow Anderson-Schulz-Flory distribution, and as a result the selectivity to LPG would be relatively limited. In particular, such a process would generally produce significant amounts of undesirable methane together with higher linear hydrocarbons.

Therefore a new synthesis method to produce LPG which overcame or at least mitigated one or more of these or other disadvantages would be desirable.

Processes exist for selectively converting syngas to for example methane or methanol. The conversion of methanol to C₂ and C₃ products as exemplified in the methanol to olefins (MTO) and methanol to propylene (MTP) is well known, for example as described in U.S. Pat. No. 6,613,951. However, in some cases, the selectivity may be limited and products may consist predominantly of C₂ and C₃ olefins.

The methanol to gasoline (MTG) process as developed by Mobil allows access to a mixed product rich in aromatics and olefins.

Neither of these processes is selective to LPG.

Recently, several investigations have been made relating to a process for the production LPG from syngas. Some investigations involve multifunctional catalyst systems. For example Zhang Q, et al. Catalysis Letters Vol 102, Nos 1-2 Jul. 2005, describes hybrid catalysts based on Pd—Ca/SiO₂ and zeolite, and on Cu—Zn/zeolite. Both hybrid catalyst systems were reported to have reasonable selectivity to LPG but the Cu—Zn/zeolite was reported to be deactivated rapidly under the high temperature reaction conditions required, and while the Pd—Ca/SiO₂ system was found to be more stable, it had a relatively low activity.

Qingjie Ge et al, Journal of Molecular Catalysis A: Chemical 278 (2007) 215-219, describes the reaction of synthesis gas to produce LPG using a mixed catalyst system in a single bed comprising a Pd—Zn—Cr methanol synthesis catalyst and a Pd-loaded zeolite for dehydration of methanol and dimethyl ether (DME). Reaction temperatures used were more than 330 degrees C. and the high reaction temperatures were reported to improve selectivity to LPG. However, despite advantageous synergy reported between the two catalysts, the lifetime of the catalyst was found to be an issue. Coking of the catalyst was thought to decrease the performance of the catalyst with time on stream. Also, the described catalyst has a Pd content of 0.5 wt %, and it would be desirable to reduce the amount of precious metal required.

A catalyst system for the production of saturated hydrocarbons, in particular C₃ and higher hydrocarbons, combining an improved selectivity and high activity with improved lifetime would be desirable.

According to an aspect of the invention there is provided an integrated process for the generation of saturated C₃ and higher hydrocarbons from carbon oxide(s) and hydrogen, the process comprising the steps of:

(a) feeding a gas feed stream including carbon oxide(s) and hydrogen to a two-stage reaction system comprising a first stage including a carbon oxide(s) conversion catalyst, where the feed stream is converted in the first stage to form an intermediate product stream,
(b) feeding the intermediate product stream to a second stage including a dehydration/hydrogenation catalyst and
(c) removing a product stream from the second stage, the product stream including saturated C₃ and higher hydrocarbons.

By separating the conversion into two reaction stages, the reaction conditions and other parameters of the two stages can be optimized independently.

The carbon oxide(s) conversion catalyst is preferably active to produce methanol in the first stage. Thus the catalyst of the first stage may include a methanol conversion catalyst. The intermediate product may therefore include methanol.

The catalyst of the second stage preferably includes a dehydration/hydrogenation catalyst. Where reference is made herein to dehydration/hydrogenation catalyst, preferably the catalyst (or hybrid catalyst) has dehydration and/or hydrogenation activity. In examples, the catalyst might be a hydrogenation catalyst in the second stage. Where the second stage has dehydration and hydrogenation activity, this may be provided by a single catalyst, by a hybrid catalyst having both dehydration and hydrogenation activity and/or by including two or more different catalyst components which may or may not be mixed or juxtaposed in the second stage.

The carbon oxide(s) conversion catalyst may be active to produce dimethyl ether (DME) in the first stage. In some examples, both methanol and DME are produced in the first stage. Thus the intermediate product stream may include DME and/or methanol.

The production of methanol from carbon oxide(s) and hydrogen is equilibrium limited. The production of DME direct from carbon oxide(s) and hydrogen is less equilibrium limited. Pressure can be used to increase the yield, as the reaction which produces methanol exhibits a decrease in volume, as disclosed in U.S. Pat. No. 3,326,956. Improved catalysts have allowed viable rates of methanol formation to be achieved at relatively low reaction temperatures, and hence allow commercial operation at lower reaction pressures. For example a CuO/ZnO/Al₂O₃ conversion catalyst may be operated at a nominal pressure of 5-10 MPa and at temperatures ranging from approximately 150 degrees C. to 300 degrees C. However, at higher reaction temperatures, reduction in catalyst lifetime has commercially been found to be a problem. A low-pressure, copper-based methanol synthesis catalyst is commercially available from suppliers such as BASF and Haldor-Topsoe. Methanol yields from copper-based catalysts are generally over 99.5% of the converted carbon oxide(s) present. Water is a by-product of the conversion of CO₂ to methanol and the conversion of synthesis gas to C₂ and C₂₊ oxygenates. In the presence of an active water gas-shift catalyst, such as a methanol catalyst or a cobalt molybdenum catalyst, the water equilibrates with the carbon monoxide to give CO₂ and hydrogen.

Recently, to seek to overcome the equilibrium limitation of the methanol synthesis catalyst, direct syngas-to-DME processes have been developed. These processes are thought to proceed via a methanol intermediate which is etherified by an added acid functionality in the catalyst, for example as described in P S Sai Prasad, et al., Fuel Processing Technology Volume 89, Issue 12, December 2008, p 1281-1286.

The conversion of methanol or DME to higher olefins may be catalysed by acidic supports such as zeolites, as exemplified in the MTO process. This reaction is characterized by its high temperatures, typically above that employed for a methanol or DME synthesis catalyst.

To produce the desired C₃ and higher hydrocarbon products, the process conditions must be suitable for chain growth from the DME to the corresponding olefins prior to hydrogenation.

By separating the two stages of the reaction system, it is possible to independently optimize the two stages. A significant advantage of this is that the methanol- and/or DME-generating catalyst can be run at conditions more suitable for improved conversion, selectivity, and/or longer catalyst life.

Preferably the first stage temperature is lower than the second stage temperature.

The temperature of the first stage may be less than 300 degrees C. Preferably, the temperature of the first stage is less than 295 degrees C., for example not more than 280 degrees C., for example not more than 250 degrees C. In examples of the invention, the temperature of the first stage may be between from about 190 to 250 degrees C., for example between from about 210 to 230 degrees C. In practical systems, it is likely that the temperature will vary across the reaction stage. Preferably the temperature of the stage is measured as an average temperature across a reaction region.

The temperature of the second stage may be more than 300 degrees C.

In some examples, the temperature of the second stage will be 320 degrees C. or more. In some examples, a temperature of 340 degrees C. or more will be preferred. In some examples the temperature of the second stage will be between from about 330 to 360 degrees C. In many cases it will be preferable for the temperature of the second stage to be less than 450 degrees C., for example less than 420 degrees C., or for example less than 400 degrees C. which may prolong the life of the catalyst. Depending on the target products, other temperatures may be used for the second stage.

The first and second stages may be operated at the same or at different pressures. Both stages may be operated for example at a pressure less than 40 bar. In some examples, it will be preferable for the second stage to be operated at a pressure lower than that of the first stage.

This feature is of particular interest and is provided independently. Thus a further aspect of the invention provides an integrated process for the generation of saturated C₃ and higher hydrocarbons from carbon oxide(s) and hydrogen, the process comprising the steps of:

(a) feeding a gas feed stream including carbon oxide(s) and hydrogen to a two-stage reaction system comprising a first stage including a carbon oxide(s) conversion catalyst, where the feed stream is converted in the first stage to form an intermediate product stream,
(b) feeding the intermediate product stream to a second stage including a dehydration/hydrogenation catalyst wherein at least a portion of the intermediate stream is converted to saturated hydrocarbons and
(c) removing a product stream from the second stage, the product stream including saturated C₃ and higher hydrocarbons,
wherein the second stage is operated at a pressure lower than that of the first stage.

In this way, a relatively high pressure could be used for the carbon oxide(s) conversion stage, for example to increase CO conversion, while hydrocarbon conversion of the second stage could be carried out at a lower pressure.

The pressure of the second stage may be not more than 1.0 MPa in some examples. For example, the second stage may be operated at a pressure of between from about 0.1 MPa to 1.0 MPa. The pressure of the second stage may be 0.1 MPa. These low pressures would be impractical for other systems which would require a higher pressure to achieve required conversion.

For example, the first stage may be operated at a pressure of less than 40 bar, less than 20 bar, or less than 10 bar. In some examples, a significantly higher pressure may be desirable.

For example, the second stage may be operated at a pressure of less than 20 bar, less than 10 bar, or less than 5 bar. In some examples, a significantly higher pressure may be desirable.

For LPG selectivity in the second stage, in some examples it will be preferable for the pressure of the second stage to be at least 1 MPa. In some examples it will be preferable for the pressure of the second stage to be less than about 2 MPa; in some examples, the selectivity of the process to methane is significant, which will be disadvantageous in many applications.

The gas hourly space velocity of the first stage may be for example between about 500 and 6000, for example between about 500 and 3000.

The gas hourly space velocity of the second stage may be for example between about 500 and 20000, for example between about 1000-10000.

Preferably the gas hourly space velocity is defined as the number of bed volumes of gas passing over the catalyst bed per hour at standard temperature and pressure.

Several configurations of the two stages are possible. An example giving less flexibility is one in which the two stages are contained within a single reactor vessel, for example as separate zones. In such a system, a heat transfer region may be provided, for example to control the reaction stage temperatures independently.

A more flexible system provides the two stages in separate vessels. At least a portion of the intermediate product stream (or effluent) exiting the first stage preferably passes directly to the second stage. Preferably, substantially all of the intermediate product stream passes to the second stage.

It will be understood that additional second stage influent components can be added to the intermediate stream upstream of the second stage. For example, addition of hydrogen and/or DME may be carried out. The intermediate stream may be subject to operations for example heat exchange upstream of the second stage and/or pressure adjustment, for example pressure reduction.

Each of the stages may include any appropriate catalyst bed type, for example fixed bed, fluidized bed, moving bed. The bed type of the first and second stages may be the same or different.

Potential application for example for the second stage is the use of a moving bed or paired bed system, for example a swing bed system, in particular where catalyst regeneration is desirable.

In preferred examples the process is a gas phase process.

The feed to the process comprises carbon oxide(s) and hydrogen. Any appropriate source of carbon oxides (for example carbon monoxide and/or carbon dioxide) and of hydrogen may be used. Processes for producing mixtures of carbon oxide(s) and hydrogen are well known. Each method has its advantages and disadvantages, and the choice of using a particular reforming process over another is normally governed by economic and available feed stream considerations, as well as by the desire to obtain the desired (H₂—CO₂):(CO+CO₂) molar ratio in the resulting gas mixture, that is suitable for further processing. Synthesis gas as used herein preferably refers to mixtures containing carbon dioxide and/or carbon monoxide with hydrogen. Synthesis gas may for example be a combination of hydrogen and carbon oxides produced in a synthesis gas plant from a carbon source such as natural gas, petroleum liquids, biomass and carbonaceous materials including coal, recycled plastics, municipal wastes, or any organic material. The synthesis gas may be prepared using any appropriate process for example partial oxidation of hydrocarbons (PDX), steam reforming (SR), advanced gas heated reforming (AGHR), microchannel reforming (as described in, for example, U.S. Pat. No. 6,284,217), plasma reforming, autothermal reforming (ATR) and any combination thereof.

A discussion of these synthesis gas production technologies is provided for in “Hydrocarbon Processing” V78, N.4, 87-90, 92-93 (April 1999) and/or “Petrole et Techniques”, N. 415, 86-93 (July-August 1998), which are both hereby incorporated by reference.

The synthesis gas source used in the present invention preferably contains a molar ratio of (H₂—CO₂):(CO+CO₂) ranging from 0.6 to 2.5. The gas composition to which the catalyst is exposed will generally differ from such a range due to for example gas recycling occurring within the reaction system. For example, in commercial methanol plants, a syngas feed molar ratio (as defined above) of 2:1 is commonly used, whereas the catalyst may experience a molar ratio of greater than 5:1 due to recycle. The gas composition experienced by the catalyst in the first stage may initially be for example between from about 0.8 to 7, for example from about 2 to 3.

Carbon oxide(s) conversion catalysts are commonly water gas shift active. The water gas shift reaction is the equilibrium of H₂ and CO₂ with CO and H₂O. The reaction conditions in the first stage preferably favour the formation of H₂ and CO₂. For the case where the carbon oxide(s) conversion catalyst is active to produce methanol, the reaction stoichiometry requires a synthesis gas molar ratio of 2:1. For the case where the carbon oxides(s) conversion catalyst is active to produce dimethyl ether (DME), the reaction coproduces water which is shifted with CO to CO₂ and hydrogen. In case, the synthesis gas molar ratio (as defined above) requirement is also 2:1 but here a reaction product is CO₂. The second stage reaction in the case of methanol synthesis in the first stage is thought to comprise initial conversion to DME and water, and subsequent conversion of DME to C₃ and higher saturated hydrocarbons and water. The second stage reaction in the case of DME synthesis in the first stage is thought to comprise only the stages of DME conversion to C₃ and higher saturated hydrocarbons and water. In this case, the product mixture additionally includes carbon dioxide.

The choice of conversion used in the first stage may impact on the choice of catalyst and/or operating conditions of the second stage. For example, a catalyst of the second stage which is water sensitive may be preferably used in combination with a DME producing catalyst in the first stage.

The carbon oxides conversion catalyst preferably comprises a methanol conversion catalyst. The carbon oxides conversion catalyst may include Cu, or Cu and Zn. For example, the catalyst of the first stage may be based on a CuO/ZnO system. The catalyst may also include a support, for example alumina.

For the case where the carbon oxide(s) conversion catalyst is active to produce methanol, preferably no additional acid co-catalyst is added.

For the case where the carbon oxide(s) conversion catalyst is active to produce DME, an acid co-catalyst is preferably added. For example, the catalyst may include a molecular sieve, or a crystalline microporous material. The catalyst may include a zeolite and/or silicoalumino phosphate (SAPO), for example a crystalline microporous silicoalumino phosphate composition. This additional co-catalyst may also for example be used as a support for the methanol catalyst. Reference is made herein to a SAPO in addition to a zeolite. Preferably, where appropriate in the context, the term zeolite as used herein may also include SAPOs.

Silicoalumino phosphates (SAPO) are known to form crystalline structures having micropores which compositions can be used as molecular sieves for example as adsorbents or catalysts in chemical reactions. SAPO materials include microporous materials having micropores formed by ring structures, including 8, 10 or 12-membered ring structures. Some SAPO compositions which have the form of molecular sieves have a three-dimensional microporous crystal framework structure of PO₂⁺, AlO₂⁻, and SiO₂ tetrahedral units. The ring structures give rise to an average pore size of from about 0.3 nm to about 1.5 nm or more. Examples of SAPO molecular sieves and methods for their preparation are described in U.S. Pat. No. 4,440,871 and U.S. Pat. No. 6,685,905 (the content of which are incorporated herein by reference). Other microporous compositions might be used. For example metal organosilicates, silicalites and/or crystalline aluminophosphates could be used.

The carbon oxide(s) conversion catalyst may comprise a copper oxide. The catalyst may further include one or more metal oxides including Cu, Zn, Ce, Zr, Al, and Cr. For example, the carbon oxide(s) conversion catalyst may comprise Cu/Zn oxides for example on alumina. For example the catalyst may comprise CuO—ZnO—Al₂O₃.

The carbon oxide(s) conversion catalyst may include an acidic support. The carbon oxide(s) conversion catalyst may include a zeolite and/or a SAPO, for example may include an acidic zeolite and/or a SAPO with stable structure like Mordenite, Y, ZSM-5, SAPO-11, SAPO-34.

The carbon oxide(s) conversion catalyst may comprise one or more of ZSM-5 and SAPO-11.

The content of the carbon oxide(s) conversion catalyst in the carbon oxide(s) conversion catalyst/M1-zeolite may be 20-80% (wt %), for example 30-60%(wt %), the percentage preferably being the ratio of the oxides to the zeolite, the measurement preferably being made for dry catalysts.

The hydrogenation catalyst may preferably include a metal, for example Pd.

Preferably the second stage includes an acidic support. Preferably the second stage includes a molecular sieve or crystalline microporous composition. The second stage may include a zeolite. For example, the zeolite may be any appropriate type, for example, Y and/or beta zeolite.

The second stage may include a SAPO, for example a crystalline microporous silicoalumino phosphate composition. The second stage may for example include a mixture of zeolite and SAPO.

Other microporous compositions might be used as the support. For example metal organosilicates, silicalites and/o crystalline aluminophosphates could be used.

A metal may also be included, for example one or more of Pd, Ru and Rh. The SAPO may include SAPO-5 and/or SAPO-37. The second stage may include for example Pd—Y, Pd-SAPO-5, Ru-SAPO-5, Pd-Beta especially for Pd—Y and Pd-SAPO-5. In many examples Cu would not be used for the second stage metal, because in examples it would not be suitable for the second stage due to its sintering at high temperature. The content of the metal in the second stage catalyst may be for example from 0.01 to 20 wt %.

The dehydration/hydrogenation catalyst may include a catalyst for conversion of methanol to C₃₊ hydrocarbons, and/or the dehydration/hydrogenation catalyst may include a catalyst for conversion of DME to C₃₊ hydrocarbons.

The catalyst for conversion of methanol and/or DME to C₃₊ hydrocarbons may comprise a Pd-modified zeolite.

The dehydration/hydrogenation catalyst may include a catalyst for conversion of DME to C₄ to C₇ hydrocarbons. The catalyst for conversion of DME to C₄ to C₇ hydrocarbons may comprise Pd-modified SAPO-5. The process may further include the step of carrying out a regeneration of catalyst of the second stage. It is known that the MTO, MTP and MTG processes require frequent regeneration of the catalysts. One source of deactivation is the build up of coke formed on the catalysts during the reaction. One way of removing such coke build up is by a controlled combustion method. Other methods include washing of the catalyst to remove the coke using for example aromatic solvent.

The regeneration of the catalyst may include heating the catalyst to a temperature of at least 500 degrees C. The temperature of the regeneration treatment may be for example at least 500 degrees C., preferably at least 550 degrees C., for example 580 degrees C. or more. It will be understood that a high temperature of treatment will be desirable to burn off the coke, but that very high temperatures will not be preferred in some cases because of the risk of reducing significantly the performance of the catalyst, for example due to metal sintering and/or zeolite thermal stability problems.

The regeneration of the catalyst used in the second stage may have added complexity where a metal is present in the catalyst as this can be affected adversely during the regeneration process. For example, the metal may sinter if a high temperature method is used. However, such sintered metals can be redispersed by an appropriate method such as treatment with carbon monoxide.

The first stage catalyst system for the synthesis of methanol and/or DME may be more sensitive to sintering than catalyst of the second stage. The separation of the two catalysts into the two stages affords the possibility of regenerating one catalyst independently of the other, Also, the reaction conditions of the two stages can be tailored for the particular catalyst system of that stage in view of for example, selectivity, lifetime, conversion and/or productivity. For example, some catalysts for conversion to methanol and/or DME are known to have excellent lifetimes under certain conditions, which are typically different from those preferred for the desired performance of the catalyst system of the second stage.

The product hydrocarbons preferably include iso-butane, wherein the proportion of iso-butane is preferably more than 60% by weight of the C₄ saturated hydrocarbons in the product. The fraction of C₄ and higher hydrocarbons produced is preferably has a high degree of branching. This can be beneficial for applications in LPG, for example giving a reduced boiling point of the C₄ fraction, and/or for C₅ and higher hydrocarbons for octane number in gasoline. In addition, the use the product LPG including propane and iso-butane as a chemical feedstock to generate the corresponding olefins is preferable in some cases to using propane and n-butane. While examples of the invention have been described herein relating to the production of LPG, in other examples, target hydrocarbons include butane (C₄) and higher hydrocarbons.

Many known syngas conversion processes are disadvantageous due to a low selectivity for the target product. One by-product which acts as a significant hydrogen sink is methane. The formation of methane can have a negative effect on the economics of the process. For example, Fischer Tropsch chemistry to produce diesel and alkanes typically produces more than 10% methane.

Preferably the molar fraction of methane in the total saturated hydrocarbons produced is less than 10%. Preferably the molar fraction of ethane in the total saturated hydrocarbons produced is less than 25%.

In some examples, the target products are C₃ and higher hydrocarbons, in particular C₄ to C₇ hydrocarbons.

A further aspect of the invention provides an apparatus for carrying out a method as defined herein.

According to a further aspect of the invention there is provided apparatus for the generation of saturated C₃ and higher hydrocarbons from a feed stream including carbon oxide(s) and hydrogen, the apparatus including a two-stage reaction system comprising:

(a) a first stage arranged to receive the feed stream and including a carbon oxide(s) conversion catalyst;
(b) a second stage arranged to receive an intermediate product stream from the first stage, the second stage including a dehydration/hydrogenation catalyst.

The carbon oxide(s) conversion catalyst may be active to produce methanol and/or DME in the first stage.

The apparatus may include at least two reaction vessels in series, including a first reaction vessel including the carbon oxides conversion catalyst, and a second reaction vessel downstream of the first including the dehydration/hydrogenation catalyst.

Each of the stages may include any appropriate catalyst bed type, for example fixed bed, fluidized bed, moving bed. The bed type of the first and second stages may be the same or different.

The carbon oxide(s) conversion catalyst may comprise a copper oxide. The carbon oxide(s) conversion catalyst may include an acidic zeolite and/or a SAPO, preferably with a stable structure such as Mordenite, Y, ZSM-5, SAPO-11, SAPO-34.

The carbon oxide(s) conversion catalyst may comprises one or more of ZSM-5 and SAPO-11.

The hydrogenation catalyst may include a source of Pd.

The second stage may include a zeolite.

The invention may further provide apparatus for the generation of saturated C₃ and higher hydrocarbons from a feed stream including carbon oxide(s) and hydrogen, the apparatus including a two-stage reaction system comprising:

(a) a first stage arranged to receive the feed stream and including a carbon oxide(s) conversion catalyst;
(b) a second stage arranged to receive an intermediate product stream from the first stage, the second stage including a dehydration/hydrogenation catalyst, wherein the apparatus means for controlling the pressure in the two-stage reaction system such that the pressure in the first stage is greater than the pressure in the second stage.

The pressure may for example be controlled using a valve configuration. For example, the apparatus may include back-pressure valves which could be used to control the pressure in the system. In an example, the apparatus could include two back-pressure valves.

Examples of the present invention provide a two-stage reaction system exhibiting a high activity (>70% CO conversion in some cases) and selectivity for LPG fraction (>70% in some cases). In some examples, coke deposition can be controlled or managed in the second stage and the selectivity to LPG may be recoverable to at least some extent by using a regeneration treatment, for example coke burning. By using a two stage reaction system, the two stages can be operated under different reaction conditions.

In examples of the two-stage reaction system, where syngas is converted to a mixture of methanol and DME at a relatively low temperature in the first stage over a Cu—ZnO—Al₂O₃/zeolite system and then converted to hydrocarbons (mainly LPG) at high temperature over a dehydration/hydrogenation catalyst, for example including a metal/zeolite in the second stage.

Such integrated process can have the characteristic that in preferred examples CO₂ emission may be less compared with a single reactor system.

The invention extends to methods and/or apparatus being substantially as herein described with reference to the accompanying drawings.

Any feature in one aspect of the invention may be applied to other aspects of the invention, in any appropriate combination. In particular, features of method aspects may be applied to apparatus aspects, and vice versa.

Preferred features of the present invention will now be described, purely by way of example, with reference to the accompanying drawings, in which:

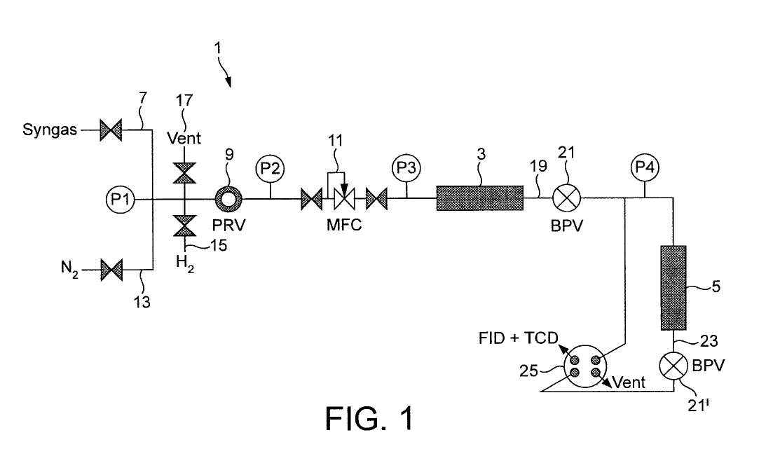

FIG. 1 shows schematically an example of a two-stage reactor system used in a process for the conversion of syngas to saturated hydrocarbons in an example of the invention;

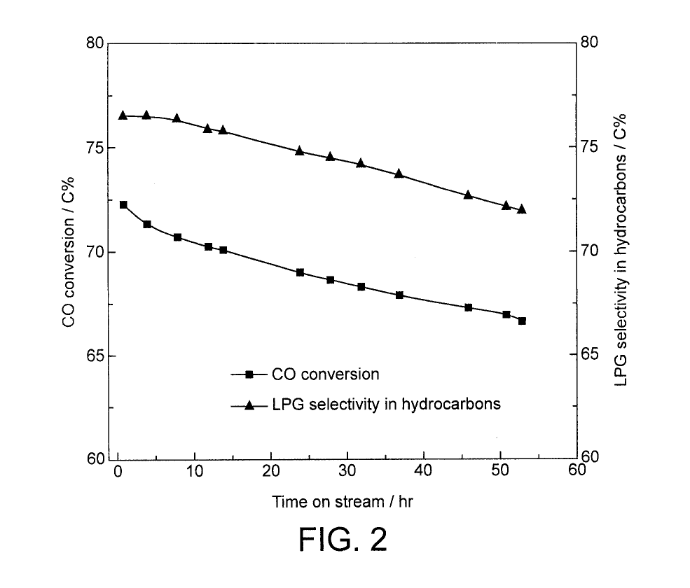

FIG. 2 shows a graph of the performance of a hybrid catalyst Cu—ZnO—Al₂O₃/Pd—Y in a one-stage reaction system of a comparative example;

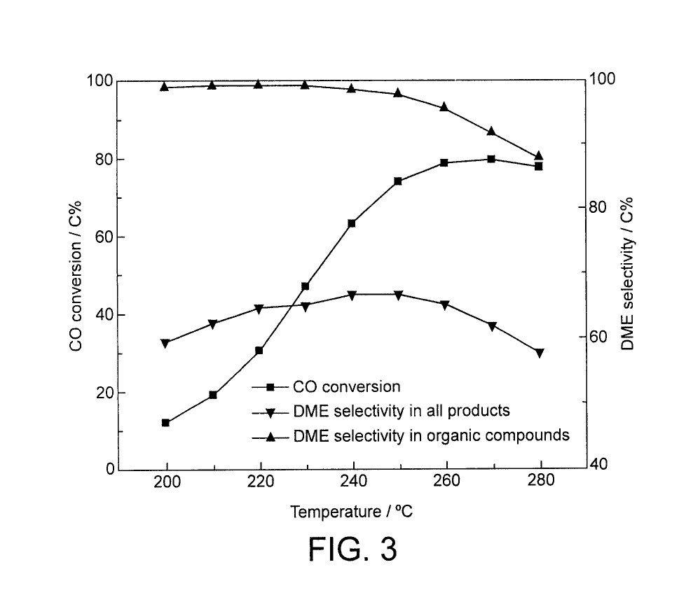

FIG. 3 shows a graph indicating the performance with temperature in the first stage of a catalyst system in a two-stage reactor system of an example;

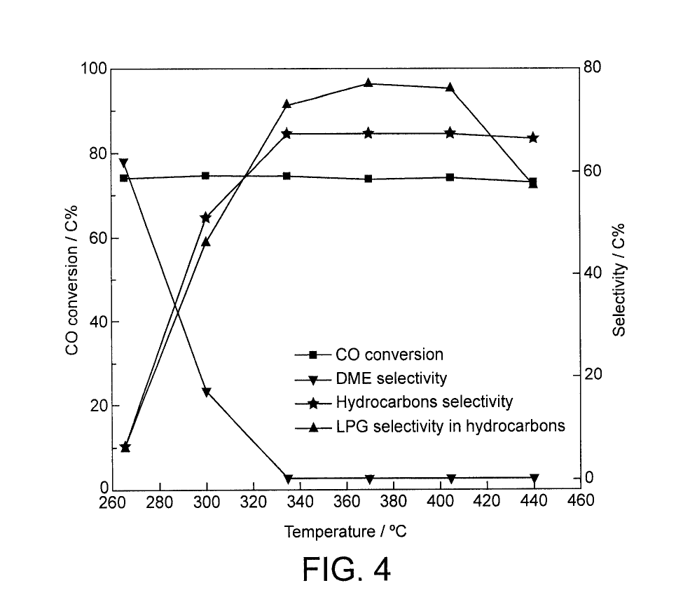

FIG. 4 shows a graph indicating the performance with temperature in the second stage of a catalyst system in a two-stage reactor system of an example;

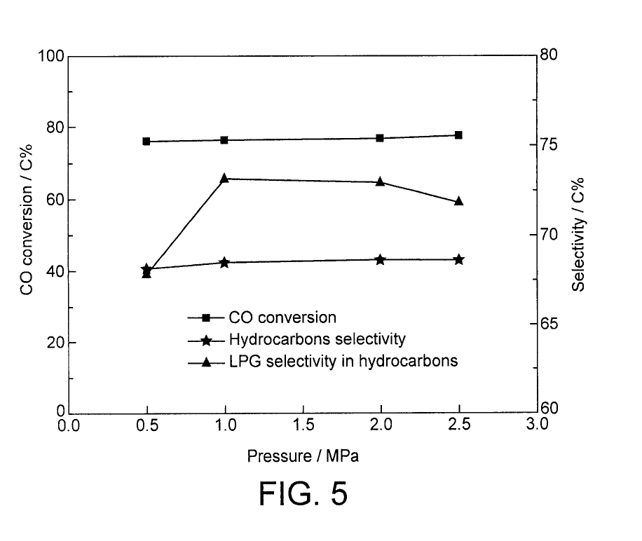

FIG. 5 shows a graph indicating the performance with pressure in the second stage of a catalyst system in a two-stage reactor system of an example; and

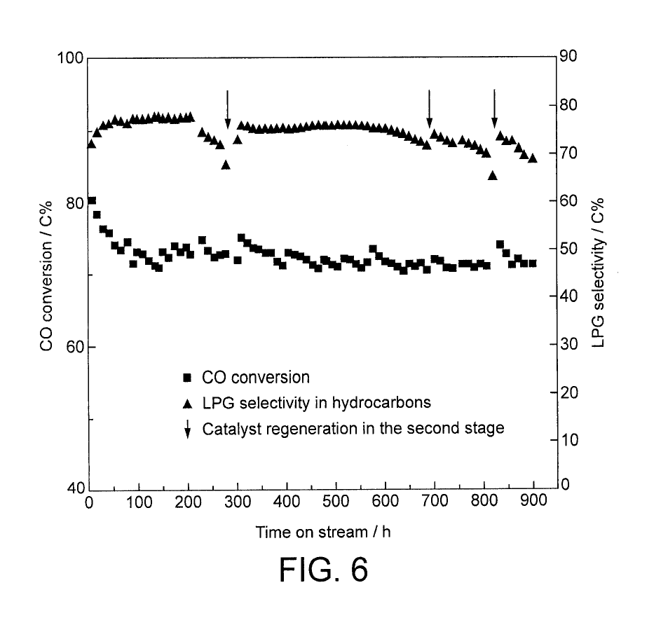

FIG. 6 shows a graph indicating the performance with time on stream for a catalyst system in a two-stage reactor system of an example.

The following describes examples catalyst systems and example methods for their preparation and describes their evaluation in a two-stage reactor system. In a comparative example, a catalyst system and method of preparation is described and the catalyst system is evaluated in a one-stage reactor system.

FIG. 1 shows schematically an example of a two-stage test reactor system 1 for LPG synthesis from syngas. The system 1 includes two reaction stages 3, 5 arranged in series. Each reaction stage 3, 5 includes a reaction vessel containing a fixed bed catalyst system. The reactions were carried out under pressurized conditions in these examples. Each stage 3, 5 was equipped with an electronic temperature controller for a furnace, a tubular reactor with an inner diameter of 10 mm, and a back pressure valve 21, 21′ downstream of the reactor. When carrying out the comparative example including a one-stage reaction, only the reactor of the first stage 3 was used.

The upstream reaction stage 3 includes a first catalyst composition including a methanol synthesis catalyst; the downstream reactor vessel 5 contains a second catalyst composition including a dehydration/hydrogenation catalyst.

A syngas feed line 7 feeds syngas via a first pressure test point P1, a pressure reducing valve 9, a second pressure test point P2, a globe valve system including a mass flowmeter 11, and a third pressure test point P3 to the first reaction stage 3. A nitrogen feed line 13 is provided for feeding N₂ to a point at the first pressure test point P1. A hydrogen feed line 15 and vent 17 is provided upstream of the pressure reducing valve 9. Intermediate product stream leaving the first reaction stage 3 via line 19 passes through a back pressure valve to a fourth pressure test point P4 before passing to the second reaction stage 5. A product stream passes from the second reaction stage 5 via line 23 through a further back pressure valve 21′.

The system further includes gas chromatography (GC) apparatus 25 arranged to receive intermediate product stream from line 19 and/or product stream from line 23. The gas chromatography apparatus 25 in this example includes a flame ionization detector (FID) and a thermal conductivity detector (TCD).

### Catalyst Evaluation

In use, the catalysts were first activated at 250 degrees C. for 5 hours in a pure hydrogen flow. Subsequently, syngas having a ratio of H₂ to CO of 2 was fed to the reaction vessels and the reaction carried out using different reaction conditions as described below. All the products from the reactor were introduced in gaseous stage and analysed by gas chromatography on-line. CO, CO₂, CH₄ and N₂ were analysed using a GC equipped with the TCD; and organic compounds were analyzed by another GC apparatus equipped with the FID.

### EXAMPLE 1

Catalyst Preparation:

A commercial Cu—ZnO—Al₂O₃ methanol synthesis catalyst (from Shenyang Catalyst Corp.) and ZSM-5 (from Nankai University Catalyst Ltd.) were powder mixed at a weight ratio of 3:1, pelletized and crushed into particles of size 20-40 mesh to form hybrid catalyst (A). This hybrid catalyst (A) was put into the first stage reactor 3 as a methanol and DME synthesis catalyst. The ratio of silica to alumina in ZSM-5 was 50. The ZSM-5 zeolite was pretreated to become proton-typed before use.

Pd modified Y zeolite (Pd—Y) was prepared by the following ion-exchange method. 10 g Y zeolite (from Nankai University Catalyst Ltd.) was added to a 200 ml solution of PdCl₂ at 60 degrees C. with stirring, and maintained for 8 h, and then washed with water, dried at 120 degrees C. and calcined at 550 degrees C. The Pd—Y was placed into the second stage reactor for methanol/DME conversion to hydrocarbons. The weight ratio of Y-zeolite to palladium in solution was 1:200. The ratio of silica to alumina in Y was 6. The Y zeolite was pretreated to become proton-typed before use.

Catalyst Evaluation:

A two-stage reaction system with fixed catalyst bed under pressurized conditions was used. The catalysts were first activated at 250 degrees C. for 5 hours in a pure hydrogen flow. Subsequently, syngas was fed to the reaction vessels and the reaction carried out using different reaction conditions as described below.

### COMPARATIVE EXAMPLE 1A

### Catalyst Preparation

The hybrid catalyst used for the comparative example one-stage reaction system was prepared by granule mixing Cu—ZnO—Al₂O₃ methanol synthesis catalyst (from Shenyang Catalyst Corp.) and Pd—Y catalyst (prepared as in Example 1) at 20-40 mesh particle size. The weight ratio of Cu—Zn—Al methanol synthesis catalyst to Pd—Y was 7:9. The ratio of silica to alumina in Y zeolite was 6.

Catalyst Evaluation:

A one-stage reaction system with fixed catalyst bed under pressurized conditions was used. The catalyst was activated at 250 degrees C. for 5 h in a pure hydrogen flow.

The catalyst was evaluated at different reaction conditions as described below.

### EXAMPLE 2

Catalyst preparation and catalyst evaluation are similar to those of Example 1, except that silicoalumino phosphate SAPO-11 (from Tianjin Chemist Scientific Ltd.) was used instead of ZSM-5 in the first stage reaction catalyst composition. The weight ratio of Cu—ZnO—Al₂O₃ to SAPO-11 is 1:1.

### EXAMPLE 3

Catalyst preparation and catalyst evaluation are similar to those of Example 2, except that the weight ratio of Cu—ZnO—Al₂O₃ to SAPO-11 is 2:1.

### EXPERIMENT 1

In comparative Example 1A, the performance of hybrid catalyst Cu—ZnO—Al₂O₃/Pd—Y in a one-stage reaction system at 280 degrees C., 2.1 MPa, GHSV=1500 h-1 to convert syngas to LPG were investigated; the results are shown in Table 1 and FIG. 2.

TABLE 1
Performance of hybrid catalyst Cu—ZnO—Al₂O₃/Pd—Y
in one-stage reaction system

| | | LPG selectivity in |
|---|---|---|
| Time (h) | CO conversion (C %) | hydrocarbons (C %) |
| 1 | 72.26 | 76.51 |
| 4 | 71.25 | 76.50 |
| 8 | 70.70 | 76.36 |
| 12 | 70.23 | 75.84 |
| 14 | 70.09 | 75.78 |
| 24 | 68.97 | 74.77 |
| 28 | 68.66 | 74.51 |
| 32 | 68.33 | 74.17 |
| 37 | 67.90 | 73.65 |
| 46 | 67.28 | 72.62 |
| 51 | 67.00 | 72.13 |
| 53 | 66.63 | 71.93 |

Table 1 shows that the hybrid catalyst Cu—ZnO—Al₂O₃/Pd—Y demonstrated relatively high activity and more than 76% selectivity for LPG at the initial stage of reaction. The conversion of CO decreased from 72% to 66% after 53 h of time on stream, and LPG selectivity dropped to 71%. Without wishing to be bound by any particular theory, it is believed that the CO conversion decreased more slowly than that previously reported in reference Catalysis Letters, 2005, 102(1-2): 51 due to the relatively low reaction temperature in comparison with the reference (335 degrees C.), but LPG selectivity dropped faster than that of the reference. This implied that the higher the reaction temperature was, the faster the Cu-based methanol synthesis catalyst deactivated; consequently, the faster CO conversion decreased. On the other hand, the high reaction temperature decreased the yield of heavy hydrocarbons (containing more than five carbon atoms) which may be deleterious for zeolite in some examples. It was identified that high reaction temperature may promote the stability of zeolite and maintain high LPG selectivity for a long time (Catalysis Letters, 2005, 102(1-2): 51). It has been identified that a difficulty of the one-stage reaction system relates to the optimization of working temperatures for Cu-based methanol synthesis catalyst and zeolite, which are absolutely different.

It has been identified in accordance with aspects of the present invention that the use of a two-stage reaction system could help to harmonize the effect of reaction temperature on Cu-based methanol synthesis catalyst and zeolite. In such systems, for example, syngas could be transformed to a mixture of methanol and DME in a first stage at a relatively low temperature (for example ≦250 degrees C.) over for example a Cu—ZnO—Al₂O₃/ZSM-5 catalyst system and then converted to hydrocarbons for example over Pd/Y in the second stage at high temperature.

### EXPERIMENT 2

The effect of reaction temperature in the first stage on DME synthesis from syngas was investigated under the pressure of 3.0 MPa over a Cu—Zn—Al/ZSM-5 catalyst of Example 1.

Reaction conditions:

Stage 1—Pressure 3.0 MPa, GHSV 2000 h-1, catalyst 0.4 g Cu—Zn—Al/ZSM-5 (3:1 by weight, powder mixing)

The results are shown in FIG. 3.

It can be seen that that the per-pass conversion of CO increased first with the increase in reaction temperature, passed through maximum of 80%, and then dropped. The variation of DME selectivity was similar to that for CO conversion. The content of DME in organic compounds in the intermediate product produced from the stage 1 reactor was more than 98% when the reaction temperature was below 250 degrees C. However, it was seen that more hydrocarbons were formed as the temperature rose from 250 to 280 degrees C. It was further seen that LPG was not the main product in the product hydrocarbons formed using this catalyst system of Cu—Zn—Al/ZSM-5. The selectivity for LPG in the hydrocarbon product was seen to gradually decrease from 49% to 27% when the temperature changed from 250 to 280 degrees C. Thus it was seen that in this example, the reaction temperature in the first stage would preferably be controlled below 250 degrees C. to increase the amount of DME, for example so that DME is the main composition of the intermediate product mixture introduced into the second stage. If different hydrocarbon products are sought, then different temperatures may be more desirable.

### EXPERIMENT 3

The catalyst system of Example 1 was used in which the first stage included 0.4 g Cu—Zn—Al/ZSM-5 and the second stage included 0.5 g Pd—Y, and the effect of temperature in the second stage on reaction performance was studied under the pressure of 2.0 MPa when the experimental conditions in the first stage were kept constant. In this example, the first stage was at a temperature of about 250 degrees C., a pressure of about 3.0 MPa, and a GHSV of about 2000 h⁻¹. The results are shown in Table 2 and FIG. 4.

TABLE 2
Effect of temperature in the second stage on reaction performance

| Temper- | CO | CO₂ | DME | Hydrocarbon | LPG |
|---|---|---|---|---|---|
| ature | conversion | selectivity | selectivity | selectivity | selectivity |
| (° C.) | (C %) | (C %) | (C %) | (C %) | (C %) |
| 265 | 74.22 | 31.35 | 62.10 | 6.55 | 6.00 |
| 300 | 74.78 | 31.73 | 17.17 | 51.10 | 46.40 |
| 335 | 74.71 | 32.20 | 0.24 | 67.56 | 73.03 |
| 370 | 73.98 | 32.33 | 0.19 | 67.48 | 77.17 |
| 405 | 74.32 | 32.5 | 0.15 | 67.35 | 76.22 |
| 440 | 73.09 | 33.32 | 0.16 | 66.52 | 57.38 |

For this experiment, LPG selectivity means LPG selectivity in hydrocarbons

Reaction Conditions:

1^{st} stage: 250 degrees C., 3.0 MPa, 2000 h⁻¹, 0.4 g Cu—Zn—Al/ZSM-5;
2^{nd} stage: 2.0 MPa, 0.5 g Pd—Y.

Table 2 indicates that CO conversion had no evident change in this example when the temperature in the second stage rose from 265 to 440 degrees C. The products at the outlet of the first stage had also been analyzed, and CO conversion calculated based on the first stage was almost the same as that based on the second stage. This indicated that the temperature of the second stage had substantially no effect on CO conversion. Also in this example, DME converted to hydrocarbons nearly totally when the temperature was higher than 335 degrees C. Meanwhile, LPG became the dominant product in hydrocarbons at higher temperatures. Therefore, in this example, an appropriate temperature for the second stage is 335-405 degrees C., in particular where LPG is a target hydrocarbon.

### EXPERIMENT 4

The catalyst system of Example 1 was used and the effect of reaction pressure in the second stage on reaction performance was studied under a second-stage temperature of 370 degrees C. when the experimental conditions in the first stage were kept constant; in this example the second stage was operated at a temperature of about 250 degrees C., a pressure of about 3.0 MPa, and GHSV of 2000 h⁻¹. The results are shown in Table 3 and FIG. 5.

TABLE 3
Effect of pressure in the second stage on reaction performance

| | CO | CO₂ | DME | Hydrocarbon | LPG |
|---|---|---|---|---|---|
| Pressure | conversion | selectivity | selectivity | selectivity | selectivity |
| (MPa) | (C %) | (C %) | (C %) | (C %) | (C %) |
| 0.5 | 76.11 | 31.85 | trace | 68.15 | 67.82 |
| 1.0 | 76.53 | 31.49 | trace | 68.51 | 73.21 |
| 2.0 | 76.88 | 31.36 | trace | 68.64 | 72.99 |
| 2.5 | 77.64 | 31.39 | trace | 68.61 | 71.85 |

LPG selectivity for this experiment means LPG selectivity in hydrocarbons

### Reaction Conditions:

1st stage: 250 degrees C., 3.0 MPa, 2000 h⁻¹, 0.4 g Cu—Zn—Al/ZSM-5;
2^{nd} stage: 370 degrees C., 0.5 g Pd—Y

Table 3 shows that CO conversion was almost unchanged when the reaction pressure in the second stage increased from 0.5 MPa to 2.5 MPa. It suggested that the pressure of the second stage had no effect on CO conversion. Hydrocarbons selectivity ascended a little as the pressure was increased. LPG selectivity went up first with the increase in reaction pressure, and then fell down. Also, a higher reaction pressure was seen to enhance the yield of methane in this experiment (3.8% at 0.5 MPa and 9.6% at 2.5 MPa). This is undesirable in these examples because methane is considered to be the most unfavorable product in this process. Therefore, it is identified that, for this experiment, a low reaction pressure, for example between about from 1.0 to 2.0 MPa is appropriate for the second stage where LPG is the desired product. If other products are favoured, other conditions may be used.

### EXPERIMENT 5

Using the catalyst system of Example 1, CO conversion and LPG selectivity in two-stage reaction system as a function of time on stream were carried out on a two-stage reaction system where:

1^{st} stage: temperature of 230-250 degrees C., pressure 3.0 MPa, GHSV 1000 h⁻¹, catalyst 0.5 g Cu—Zn—Al/ZSM-5;

2^{nd} stage: temperature 350 degrees C., pressure 1.0 MPa, catalyst 0.5 g Pd—Y.

The results are shown in Table 4 and in FIG. 6.

FIG. 6 shows CO conversion and LPG selectivity in a two-stage reaction system as a function of time on stream. CO conversion was seen to decrease from 80% to 71% during the initial 72 hours of the experiment at the initial temperature of 230 degrees C., and was kept to a level of higher than 71% throughout this experiment by the gradual increase of temperature in the first stage from 230 degrees C. to 250 degrees C. Without wishing to be bound by any particular theory, the increase of reaction temperature for keeping the CO conversion stable was thought to imply a slow deactivation of Cu—Zn—Al/ZSM-5 catalyst in stage 1; this was thought to be due at least in part to the sintering of Cu.

TABLE 4
The performance of two-stage reaction system
as a function of time on stream

| | CO | CO₂ | DME | Hydrocarbon | LPG |
|---|---|---|---|---|---|
| Time | conversion | selectivity | selectivity | selectivity | selectivity |
| (h) | (C %) | (C %) | (C %) | (C %) | (C %) |
| 4 | 81.59 | 31.38 | 0.03 | 68.59 | 66.95 |
| 8 | 80.34 | 31.21 | Trace | 68.79 | 72.12 |
| 20 | 78.34 | 31.10 | Trace | 68.90 | 74.50 |
| 40 | 75.85 | 31.10 | Trace | 68.90 | 76.52 |
| 60 | 73.67 | 31.26 | Trace | 68.74 | 77.04 |
| 200 | 73.8 | 30.47 | Trace | 69.53 | 77.54 |
| 220 | 76.76 | 30.66 | Trace | 69.34 | 74.96 |
| 240 | 73.53 | 30.64 | Trace | 69.36 | 73.99 |
| 260 | 72.18 | 30.63 | Trace | 69.37 | 72.54 |
| 276 | 73.96 | 30.35 | Trace | 69.65 | 69.50 |
| 280 | 72.93 | 30.45 | Trace | 69.55 | 67.83 |
| 304^{R} | 72.02 | 30.06 | Trace | 69.94 | 72.91 |
| 308 | 76.11 | 30.61 | Trace | 69.39 | 75.48 |
| 400 | 71.97 | 30.61 | 0.07 | 69.32 | 75.50 |
| 420 | 72.79 | 30.33 | 0.09 | 69.58 | 75.37 |
| 440 | 72.16 | 30.47 | 0.09 | 69.44 | 75.56 |
| 500 | 71.28 | 30.17 | 0.11 | 69.72 | 75.9 |
| 600 | 72.04 | 29.70 | 0.11 | 70.19 | 75.22 |
| 620 | 71.14 | 29.40 | 0.17 | 70.43 | 74.58 |
| 640 | 70.46 | 29.73 | 0.08 | 70.19 | 74.16 |
| 660 | 71.22 | 29.67 | 0.08 | 70.25 | 73.03 |
| 692 | 71.77 | 29.72 | 0.08 | 70.20 | 71.33 |
| 708^{R} | 71.76 | 29.89 | 0.05 | 70.06 | 73.77 |
| 720 | 71.54 | 29.93 | 0.07 | 70.00 | 73.12 |
| 740 | 70.77 | 29.89 | 0.14 | 69.97 | 72.05 |
| 748 | 72.21 | 29.67 | Trace | 70.33 | 72.52 |
| 760 | 71.34 | 29.36 | 0.08 | 70.56 | 72.64 |
| 780 | 70.89 | 29.72 | 0.12 | 70.16 | 71.58 |
| 800 | 71.21 | 29.67 | 0.18 | 70.15 | 70.64 |
| 824 | 77.65 | 33.00 | 0.03 | 66.97 | 64.94 |
| 836^{R} | 73.95 | 29.60 | Trace | 70.40 | 73.38 |
| 840 | 73.45 | 29.63 | 0.02 | 70.35 | 73.01 |
| 860 | 71.21 | 29.38 | 0.05 | 70.57 | 72.44 |
| 872 | 72.02 | 29.47 | 0.12 | 70.41 | 70.89 |
| 884 | 71.38 | 30.81 | 0.05 | 69.14 | 69.59 |
| 896 | 72.30 | 31.66 | 0.03 | 68.31 | 70.13 |

Note:
LPG selectivity in this experiment means LPG selectivity in hydrocarbons;
^{R}means regeneration of Pd—Y in the second stage.

### Reaction Conditions:

1^{st} stage: 230 to 250 degrees C., 3.0 MPa, 1000 h⁻¹, 0.5 g Cu—Zn—Al/ZSM-5;
2^{nd} stage: 350 degrees C., 1.0 MPa, 0.5 g Pd—Y

Pd—Y in the second stage was seen to demonstrate high LPG selectivity of 78% after the initial activation period, and then dropped to 65% after 300 hours on stream. Characterisation using temperature programmed oxidation with mass spectrometry (TPO-MS) was used for Pd—Y. Distinct TPO-MS profiles of Pd—Y before and after reaction showed CO₂ peaks at 489 and 572 degrees C. and H₂O peaks. Without wishing to be bound by any particular theory, it was thought that the peaks could be attributed to the burning of coke retained in the Pd—Y and that the retained coke could be divided into two groups. One included aliphatic hydrocarbons with relatively high H/C ratio burning at 489 degrees C. and the other included aromatic hydrocarbons with low H/C ratio releasing plenty of CO₂ and a little H₂O at 572 degrees C. It was further understood that the presence of the retained coke was deleterious for activity and selectivity of the Pd—Y.

In the present experiment, the catalyst was heated in a “regeneration” treatment periodically. The regeneration in this experiment included coke burning with a 5% O₂/95% Ar gaseous mixture until there was no CO₂ detected by TCD. For example, the O₂/Ar mixture could be introduced to the apparatus upstream of the first pressure reducing valve 9. The temperature of the regeneration treatment in this example was 580 degrees C. In this example, a regeneration treatment was carried out after 300 hours, 700 hours and 832 hours, as indicated by the arrows in the graph of FIG. 6.

In this experiment, and as can be seen from the results, the selectivity of the Pd—Y catalyst to LPG was recovered to some extent after each regeneration treatment.

The decrease of LPG selectivity was mainly attributed to coke deposition, and could be recovered to a great extent by coke burning at high temperature.

### EXPERIMENT 6

The catalyst system of Example 2 was used, and only the first stage catalyst was evaluated at first stage reaction conditions of

Temperature: 250 degrees C.,

Pressure: 4.0 MPa

GHSV: 1000 h⁻¹.

The results show that CO conversion could achieve 69.5% with a selectivity of 70.1% for DME and selectivity of 0.4% for methanol using Cu—ZnO—Al₂O₃/SAPO-11 catalyst.

### EXPERIMENT 7

The catalyst system of Example 3 was used and the catalysts were evaluated at the reaction conditions of:

1^{st} stage: temperature 260 degrees C., pressure 3.0 MPa, GHSV 2000 h⁻¹.

2^{nd} stage: temperature 335 degrees C., pressure 1.5 MPa.

The results show that CO conversion could achieve 59.5% with a selectivity of 69.0% for LPG in hydrocarbons using Cu—ZnO—Al₂O₃/SAPO-11 catalyst.

### EXAMPLE 4

### Catalyst Preparation

A commercial Cu—ZnO—Al₂O₃ methanol synthesis catalyst (from Shenyang Catalyst Corp.) was put into the first stage reactor as methanol synthesis catalyst.

Pd modified Y zeolite (Pd—Y) was prepared by the following ion-exchange method. 10 g Y zeolite (from Nankai University Catalyst Ltd.) was added to a 200 ml solution of PdCl₂ at 60 degrees C. with stirring, and maintained for 8 h, and then washed with water, dried at 120 degrees C. and calcined at 550 degrees C. The Pd—Y was placed into the second stage reactor for methanol conversion to hydrocarbons. The weight ratio of palladium in solution to Y-zeolite was 1:200. The ratio of silica to alumina in Y was 6. The Y zeolite was pretreated to become proton-typed before use.

### Catalyst Evaluation:

A two-stage reaction system with fixed catalyst bed under pressurized conditions was used. The catalysts were first activated at 250 degrees C. for 5 hours in a pure hydrogen flow. Subsequently, syngas was fed to the reaction vessels and the reaction carried out using different reaction conditions as described below.

### EXAMPLE 5

Catalyst preparation and catalyst evaluation are similar to those of Example 4, except that Pd-SAPO-5 was used instead of Pd—Y in the second stage. Pd-modified SAPO-5 was prepared using an ion-exchange method. For example, a Pd-modified SAPO-5 was prepared by the following method. 10 g SAPO-5 (synthesized according to reported methods, for example Wang L et al, Microporous and Mesoporous Materials, 2003, Vol 64, 63˜68) was added to a 200 ml solution of PdCl₂ at 60° C. with stirring, and maintained for 8 h, and then washed with water, dried at 120° C. and calcined at 550° C.

### EXPERIMENT 8

The catalyst system of Example 4 was used in which the first stage included 1.0 g Cu—Zn—Al and the second stage included 0.5 g Pd—Y. In this example, the first stage was at a temperature of about 210 degrees C., a pressure of about 3.0 MPa, and a GHSV of about 1500 h⁻¹. The second stage was at a temperature of about 335 degrees C., a pressure of about 1.0 MPa. The results are shown in Table 5.

TABLE 5
Hydrocarbon synthesis via methanol from syngas in two-stage reaction system

| CO conversion | Selectivity (C %) | | | | Hydrocarbon distribution (C %) | | | | | |
|---|---|---|---|---|---|---|---|---|---|---|
| (C %) | CO₂ | DME | CH₃OH | HCs | C₁ | C₂ | C₃ | C₄ | C₅ | C₆₊ |
| 12.1 | 3.1 | 0 | 0 | 96.9 | 4.1 | 17.0 | 34.3 | 34.7 | 7.5 | 2.4 |

HCs means hydrocarbons. C₃-C₄ selectivity in hydrocarbon is higher than 69.0%.

### EXPERIMENT 9

The catalyst system of Example 4 was used in which the first stage included 2.0 g Cu—Zn—Al and the second stage included 0.5 g Pd—Y. In this example, the first stage was at a temperature of about 220 degrees C., a pressure of about 3.0 MPa, and a GHSV of about 500 h⁻¹. The second stage was at a temperature of about 320 degrees C., a pressure of about 1.0 MPa. The results are shown in Table 6.

TABLE 6
Hydrocarbon synthesis via methanol from syngas in two-stage reaction system

| CO conversion | Selectivity (C %) | | | | Hydrocarbon distribution (C %) | | | | | |
|---|---|---|---|---|---|---|---|---|---|---|
| (C %) | CO₂ | DME | CH₃OH | HCs | C₁ | C₂ | C₃ | C₄ | C₅ | C₆₊ |
| 72.7 | 4.1 | 0.2 | 1.4 | 94.3 | 17.5 | 20.4 | 41.9 | 17.0 | 2.6 | 0.6 |

HCs means hydrocarbons. C₃-C₄ selectivity in hydrocarbon is higher than 58.9%.

### EXPERIMENT 10

The catalyst system of Example 4 was used in which the first stage included 2.0 g Cu—Zn—Al and the second stage included 1.0 g Pd—Y. In this example, the first stage was at a temperature of about 220 degrees C., a pressure of about 3.0 MPa, and a GHSV of about 1000 h⁻¹. The second stage was at a temperature of about 320 degrees C., a pressure of about 0.1 MPa. The results are shown in Table 7.

TABLE 7
Hydrocarbon synthesis via methanol from syngas in two-stage reaction system

| CO conversion | Selectivity (C %) | | | | Hydrocarbon distribution (C %) | | | | | |
|---|---|---|---|---|---|---|---|---|---|---|
| (C %) | CO₂ | DME | CH₃OH | HCs | C₁ | C₂ | C₃ | C₄ | C₅ | C₆₊ |
| 56.2 | 3.7 | 0.2 | 0 | 96.1 | 3.3 | 11.9 | 41.1 | 35.3 | 6.1 | 2.3 |

HCs means hydrocarbons. C₃-C₄ selectivity in hydrocarbon is higher than 76.4%.

### EXPERIMENT 11

The catalyst system of Example 4 was used in which the first stage included 2.0 g Cu—Zn—Al and the second stage included 1.0 g Pd—Y. In this example, the first stage was at a temperature of about 220 degrees C., a pressure of about 4.5 MPa, and a GHSV of about 1500 h⁻¹. The second stage was at a temperature of about 320 degrees C., a pressure of about 0.1 MPa. The results are shown in Table 8.

TABLE 8
Hydrocarbon synthesis via methanol from syngas in two-stage reaction system

| CO conversion | Selectivity (C %) | | | | Hydrocarbon distribution (C %) | | | | | |
|---|---|---|---|---|---|---|---|---|---|---|
| (C %) | CO₂ | DME | CH₃OH | HCs | C₁ | C₂ | C₃ | C₄ | C₅ | C₆₊ |
| 47.7 | 2.6 | 0.2 | 0 | 97.2 | 2.5 | 9.4 | 39.7 | 38.6 | 6.9 | 2.9 |

HCs means hydrocarbons. C₃-C₄ selectivity in hydrocarbon is higher than 78.3%.

### EXPERIMENT 12

The catalyst system of Example 4 was used in which the first stage included 1.0 g Cu—Zn—Al and the second stage included 0.5 g Pd—Y. In this example, the first stage was at a temperature of about 220 degrees C., a pressure of about 3.0 MPa, and a GHSV of about 1000 h⁻¹. The second stage was at a temperature of about 320 degrees C., a pressure of about 1.0 MPa. The results are shown in Table 9.

TABLE 9
Hydrocarbon synthesis via methanol from syngas in two-stage reaction system

| Time | CO conversion | Selectivity (C %) | | | | Hydrocarbon distribution (C %) | | | | | |
|---|---|---|---|---|---|---|---|---|---|---|---|
| (h) | (C %) | CO₂ | DME | CH₃OH | HCs | C₁ | C₂ | C₃ | C₄ | C₅ | C₆₊ |
| 1 | 38.4 | 5.8 | 0.2 | 0.1 | 93.9 | 10.1 | 17.0 | 45.0 | 22.8 | 3.8 | 1.4 |
| 8 | 40.7 | 4.5 | 1.0 | 0.4 | 94.1 | 8.6 | 10.0 | 36.7 | 35.6 | 6.6 | 2.4 |
| 20 | 34.3 | 3.9 | 0.8 | 0.3 | 95.0 | 5.1 | 10.6 | 34.1 | 39.7 | 7.3 | 3.2 |
| 40 | 31.6 | 3.5 | 0.7 | 0.3 | 95.5 | 3.5 | 12.3 | 33.3 | 40.0 | 7.7 | 3.2 |
| 50 | 31.1 | 3.3 | 0.7 | 0.3 | 95.7 | 3.0 | 13.1 | 31.7 | 40.6 | 8.3 | 3.3 |

HCs means hydrocarbons. C₃-C₄ selectivity in hydrocarbon is higher than 72%.

### EXPERIMENT 13

The catalyst system of Example 5 was used in which the first stage included 1.0 g Cu—Zn—Al and the second stage included 0.5 g Pd-SAPO-5. In this example, the first stage was at a temperature of about 220 degrees C., a pressure of about 3.0 MPa, and a GHSV of about 1000 h⁻¹. The second stage was at a temperature of about 320 degrees C., a pressure of about 1.0 MPa. The results are shown in Table 10.

TABLE 10
Hydrocarbon synthesis via methanol from syngas in two-stage reaction system

| CO con. | Selectivity (C %) | | | | Hydrocarbon distribution (C %) | | | | | | | |
|---|---|---|---|---|---|---|---|---|---|---|---|---|
| (C %) | CO₂ | DME | CH₃OH | HCs | C₁ | C₂ | C₃ | C₄ | C₅ | C₆ | C₇ | C₈₊ |
| 38.8 | 3.7 | 0.2 | 0 | 96.1 | 4.1 | 3.5 | 13.0 | 53.2 | 14.8 | 7.1 | 2.0 | 2.3 |

HCs means hydrocarbons. C₄-C₇ selectivity in hydrocarbon is 77.1%.

It will be understood that the present invention has been described above purely by way of example, and modification of detail can be made within the scope of the invention.

Each feature disclosed in the description, and (where appropriate) the claims and drawings may be provided independently or in any appropriate combination.

1-46. (canceled)

47. An integrated process for the generation of saturated C₃ and higher hydrocarbons from carbon oxide(s) and hydrogen, the process comprising the steps of:
(a) feeding a gas feed stream including carbon oxide(s) and hydrogen to a two-stage reaction system comprising a first stage including a carbon oxide(s) conversion catalyst, where the feed stream is converted in the first stage to form an intermediate product stream,
(b) feeding the intermediate product stream to a second stage including a dehydration/hydrogenation catalyst wherein at least a portion of the intermediate stream is converted to saturated hydrocarbons and
(c) removing a product stream from the second stage, the product stream including saturated C₃ and higher hydrocarbons,
wherein the second stage is operated at a pressure lower than that of the first stage.

48. A process according to claim 47, wherein the pressure of the second stage is not more than 1.0 MPa.

49. A process according to claim 47 wherein the carbon oxide(s) conversion catalyst is active to produce methanol and/or dimethyl ether (DME) in the first stage.

50. A process according to claim 47 wherein the temperature of the first stage less than 300 degrees C.

51. A process according to claim 47 wherein the temperature of the second stage is more than 300 degrees C.

52. A process according to claim 47 wherein the carbon oxide(s) conversion catalyst comprises a copper oxide.

53. A process according to claim 47 wherein the carbon oxide(s) conversion catalyst comprises a methanol synthesis catalyst and/or DME synthesis catalyst.

54. A process according to claim 53, wherein the catalyst comprises one or more from the group comprising Cu—ZnO—Al₂O₃, Cu—ZnO—Al₂O₃/HZSM-5, and Cu—ZnO—Al₂O₃/SAPO-11.

55. A process according to claim 47 wherein the carbon oxide(s) conversion catalyst includes a zeolite and/or a SAPO.

56. A process according to claim 47 wherein the carbon oxide(s) conversion catalyst includes an acidic zeolite selected from the group comprising Mordenite, Y-zeolite and ZSM-5.

57. A process according to claim 47 wherein the carbon oxide(s) conversion catalyst includes a SAPO selected from SAPO-11 and SAPO-34.

58. A process according to claim 53 wherein the carbon oxide(s) conversion catalyst comprises one or more of ZSM-5 and SAPO-11.

59. A process according to claim 47 wherein the hydrogenation catalyst includes a source of Pd.

60. process according to claim 47 wherein the second stage includes a zeolite.

61. A process according to claim 47 wherein the second stage includes a SAPO.

62. A process according to claim 47 wherein the dehydration/hydrogenation catalyst includes a catalyst for conversion of methanol to C₃₊ hydrocarbons.

63. A process according to claim 47 wherein the dehydration/hydrogenation catalyst includes a catalyst for conversion of DME to C₃₊ hydrocarbons.

64. A process according to claim 62 wherein the catalyst for conversion of methanol and/or DME to C₃₊ hydrocarbons comprises a Pd-modified zeolite.

65. A process according to claim 63, including a catalyst for conversion of DME to C₄ to C₇ hydrocarbons.

66. A process according to claim 65, wherein the catalyst for conversion of DME to C₄ to C₇ hydrocarbons comprises Pd-modified SAPO-5.

67. A process according to claim 47 further including the step of carrying out a regeneration of catalyst of the second stage.

68. A process according to claim 67, wherein the regeneration of the catalyst includes heating the catalyst to a temperature of at least 500 degrees C.

69. A process according to claim 47 wherein product hydrocarbons include iso-butane, wherein the proportion of iso-butane is more than 60% by weight of the C₄ saturated hydrocarbons in the product.

70. A process according to claim 47 wherein the molar fraction of methane in the total saturated hydrocarbons produced is less than 10%.

71. Apparatus for the generation of saturated C₃ and higher hydrocarbons from a feed stream including carbon oxide(s) and hydrogen, the apparatus including a two-stage reaction system comprising:
(a) a first stage arranged to receive the feed stream and including a carbon oxide(s) conversion catalyst;
(b) a second stage arranged to receive an intermediate product stream from the first stage, the second stage including a dehydration/hydrogenation catalyst,
wherein the apparatus means for controlling the pressure in the two-stage reaction system such that the pressure in the first stage is greater than the pressure in the second stage.

72. Apparatus according to claim 71, wherein the pressure is controlled using a valve configuration.

73. Apparatus according to claim 72, wherein the carbon oxide(s) conversion catalyst is active to produce methanol and/or DME in the first stage.

74. Apparatus according to claim 73 wherein the apparatus includes at least two reaction vessels in series, including a first reaction vessel including the carbon oxides conversion catalyst, and a second reaction vessel downstream of the first including the hydrogenation catalyst.

75. Apparatus according to claim 73 wherein the carbon oxide(s) conversion catalyst comprises a copper oxide.

76. Apparatus according to claim 73 wherein the carbon oxide(s) conversion catalyst includes a zeolite and/or a SAPO.

77. Apparatus according to claim 73 wherein the carbon oxide(s) conversion catalyst includes an acidic zeolite selected from the group comprising Mordenite, Y-zeolite and ZSM-5.

78. Apparatus according to claim 73 wherein the carbon oxide(s) conversion catalyst includes a SAPO selected from SAPO-11 and SAPO-34.

79. Apparatus according to claim 77 wherein the carbon oxide(s) conversion catalyst comprises one or more of ZSM-5 and SAPO-11.

80. Apparatus according to claim 73 wherein the hydrogenation catalyst includes a source of Pd.

81. Apparatus according to claim 73 wherein the second stage includes a zeolite.

82. An integrated process for the generation of saturated C₃ and higher hydrocarbons from carbon oxide(s) and hydrogen, the process comprising the steps of:
(a) feeding a gas feed stream including carbon oxide(s) and hydrogen to a two-stage reaction system comprising a first stage including a carbon oxide(s) conversion catalyst, where the feed stream is converted in the first stage to form an intermediate product stream,
(b) feeding the intermediate product stream to a second stage including a dehydration/hydrogenation catalyst wherein at least a portion of the intermediate stream is converted to saturated hydrocarbons and
(c) removing a product stream from the second stage, the product stream including saturated C₃ and higher hydrocarbons,

83. A process according to claim 82, wherein the carbon oxide(s) conversion catalyst is active to produce methanol and/or dimethyl ether (DME) in the first stage.

84. A process according to claim 83 wherein the carbon oxide(s) conversion catalyst comprises a methanol synthesis catalyst and/or DME synthesis catalyst.

85. A process according to claim 84, wherein the catalyst comprises one or more from the group comprising Cu—ZnO—Al₂O₃, Cu—ZnO—Al₂O₃/HZSM-5, and Cu—ZnO—Al₂O₃/SAPO-11.

86. A process according to claim 83 wherein the dehydration/hydrogenation catalyst includes a catalyst for conversion of methanol to C₃₊ hydrocarbons.

87. A process according to claim 83 wherein the dehydration/hydrogenation catalyst includes a catalyst for conversion of DME to C₃₊ hydrocarbons.

88. A process according to claim 87 wherein the catalyst for conversion of methanol and/or DME to C₃₊ hydrocarbons comprises a Pd-modified zeolite.

89. A process according to claim 88, including a catalyst for conversion of DME to C₄ to C₇ hydrocarbons.

90. A process according to claim 89, wherein the catalyst for conversion of DME to C₄ to C₇ hydrocarbons comprises Pd-modified SAPO-5.

91. Apparatus for the generation of saturated C₃ and higher hydrocarbons from a feed stream including carbon oxide(s) and hydrogen, the apparatus including a two-stage reaction system comprising:
(a) a first stage arranged to receive the feed stream and including a carbon oxide(s) conversion catalyst;
(b) a second stage arranged to receive an intermediate product stream from the first stage, the second stage including a dehydration/hydrogenation catalyst.
